(19) United States
(12) Patent Application Publication
(10) Pub. No.: US 2026/0069569 A1
(43) Pub. Date: Mar. 12, 2026

(54) EBSELEN CONTAINING ORAL DOSAGE FORMS

(71) Applicant: SOUND PHARMACEUTICALS INC., Seattle, WA (US)

(72) Inventors: Jonathan KIL, Seattle, WA (US); G. Michael WALL, Fort Worth, TX (US)

(21) Appl. No.: 18/872,701

(22) PCT Filed: Jun. 6, 2023
(86) PCT No.: PCT/US2023/068009

§ 371 Date: Dec. 6, 2024

Related U.S. Application Data
(60) Provisional application No. 63/349,470, filed on Jun. 6, 2022.

(51) Int. Cl. A61K 31/41; A61K 9/00; A61K 9/20; A61K 9/48; A61K 47/12

(52) U.S. Cl. CPC A61K 31/41; A61K 9/0053; A61K 9/2009; A61K 9/2013; A61K 9/2054; A61K 9/4833; A61K 9/485; A61K 9/4858; A61K 9/4866; A61K 47/12

(57) ABSTRACT

The present disclosure provides a pharmaceutical composition comprising ebselen, wherein the composition is formulated for oral administration. Also provided are pharmaceutical dosage forms including the same. In some embodiments, the dosage form is a solid dosage form, such as a tablet (e.g., an uncoated pressed tablet). Also provided are methods of delivering the subject ebselen pharmaceutical dosage forms to a subject to achieve an enhanced maximum blood plasma concentration (Cₘₐₓ) with respect to a control ebselen formulation, and a maximum blood plasma concentration (Cₘₐₓ) for ebselen within 4 hours or less of administration.

### 1. INTRODUCTION

Ebselen (2-Phenyl-1,2-benzoselenazol-3(2H)-one) is an anti-inflammatory compound with glutathione peroxidase (GPx1) activity of the following structure:

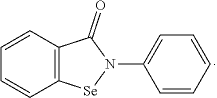 [SMILES: O=c1c2ccccc2[se]n1-c1ccccc1]

GPx1 is the dominant catalytic antioxidant enzyme in the mammalian inner ear, and its activity is decreased after noise or ototoxic insult. Several preclinical and clinical studies have demonstrated the safety and efficacy of SPI-1005 (ebselen) at preventing and treating different forms of sensorineural hearing loss. SPI-1005 capsules, containing 200 mg ebselen, have demonstrated the potential for a safe, well tolerated, oral treatment for Meniere's Disease, a disease for which there are no FDA-approved treatments. Ebselen treatment has been shown to prevent or reverse the pathologic changes in the cochlea following noise- or ototoxin-induced injury, resulting in improved physiology measured by several types of auditory stimuli. Ebselen treatment has been shown to have use in the treatment and/or prevention of chemotherapy induced ototoxicity, and treatment and/or prevention of aminoglycoside-induced ototoxicity in patients with cystic fibrosis. Ebselen treatment has further been shown to have use in treating acute lung infections and related conditions or diseases, such as coronavirus-mediated lung injuries.

Improved oral dosage forms including ebselen are of interest.

### 2. SUMMARY

The present disclosure provides a pharmaceutical composition comprising ebselen, wherein the composition is formulated for oral administration. Also provided are pharmaceutical dosage forms including the same. In some embodiments, the dosage form is a solid dosage form, such as a tablet (e.g., an uncoated pressed tablet). Also provided are methods of delivering the subject ebselen pharmaceutical dosage forms to a subject to achieve an enhanced maximum blood plasma concentration (Cₘₐₓ) with respect to a control ebselen formulation, and a maximum blood plasma concentration (Cₘₐₓ) for ebselen within 4 hours or less of administration.

A first aspect of this disclosure includes a pharmaceutical composition comprising: 40-60% w/w ebselen; 20-40% w/w filler; 2-9% disintegrant; 0.1-1% lubricant; and 0.1-1% glidant (e.g., colloidal silica dioxide), wherein the composition is formulated for oral administration.

A second aspect of this disclosure includes a pharmaceutical dosage form including a pharmaceutical composition comprising 40-60% w/w ebselen; 20-40% w/w filler; 2-9% disintegrant; 0.1-1% lubricant; and 0.1-1% glidant (e.g., colloidal silica dioxide) (e.g., as described herein).

A third aspect of this disclosure includes a method of delivering a therapeutically effective amount of ebselen to a subject in need thereof, comprising orally administering to a subject in need thereof a subject pharmaceutical dosage form (e.g., as described herein), to achieve: an enhanced maximum blood plasma concentration (Cₘₐₓ) for ebselen that is greater than that achieved with a control ebselen formulation; and a maximum blood plasma concentration (Cₘₐₓ) for ebselen that is achieved within 4 hours or less of administration.

A fourth aspect of this disclosure includes an uncoated pressed tablet comprising 400 mg ebselen, 265 mg microcrystalline cellulose, 28 mg croscarmellose sodium, 3.5 mg magnesium stearate, and 3.5 mg colloidal silicon dioxide.

### 3. BRIEF DESCRIPTION OF THE DRAWINGS

These and other features, aspects, and advantages of the present invention will become better understood with regard to the following description, and accompanying drawings where:

FIG. 1 shows the dissolution profile of Formulation 2 (ebselen tablet 400 mg, top line) versus Formulation 1 (a control ebselen dosage from, capsule 200 mg, bottom two lines).

### 4. DETAILED DESCRIPTION

As summarized above, this disclosure provides a pharmaceutical composition comprising ebselen, wherein the composition is formulated for oral administration. Also provided are pharmaceutical dosage forms including the same. In some embodiments, the dosage form is a solid dosage form, such as a tablet (e.g., an uncoated pressed tablet). Also provided are methods of delivering the subject ebselen pharmaceutical dosage forms to a subject to achieve an enhanced maximum blood plasma concentration (Cₘₐₓ) with respect to a control ebselen formulation, and a maximum blood plasma concentration (Cₘₐₓ) for ebselen within 4 hours or less of administration.

The pharmaceutical compositions of this disclosure comprising ebselen are described in greater detail below. Also described are pharmaceutical dosage forms comprising ebselen, and methods for delivering the same to a subject.

### 4.1. Pharmaceutical Compositions

As summarized above, this disclosure provides a pharmaceutical composition comprising ebselen, wherein the composition is formulated for oral administration.

### 4.1.1. Ebselen

The subject pharmaceutical compositions include ebselen (2-Phenyl-1,2-benzoselenazol-3(2H)-one), an anti-inflammatory compound with glutathione peroxidase (GPx1) activity of the following structure:

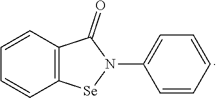 [SMILES: O=c1c2ccccc2[se]n1-c1ccccc1]

Ebselen (International Non-proprietary Name (INN)) is also known as 2-phenyl-1,2-benzisoselenazol-3(2H)-one (chemical name), 60940-34-3 (Chemical Abstracts Service (CAS) Number), SPI-1005, C05110703, C05110703-B, N0285 (Sound Pharmaceuticals, Inc., code numbers), PZ 51 (A. Natterman & Cie GmbH (Cologne, F.R.G.) Code Number), DR3305 (Watanabe, 1997), ebselene, ebselenum, ebseleno and harmokisane.

In some embodiments, the subject pharmaceutical composition comprises 45-60% w/w ebselen. In some embodiments, the subject pharmaceutical composition comprises 50-60% w/w ebselen, such as 50%, 51%, 52%, 53%, 54%, 55%, 56%, 57%, 58%, 59%, or 60% w/w ebselen.

In some embodiments, the pharmaceutical composition comprises from about 30% to about 80% w/w ebselen. In further embodiments, the pharmaceutical composition comprises from about 30% to about 75% w/w, or from about 30% to about 70% w/w, about 30% to about 65% w/w, about 30% to about 60%, about 40% to 60%, or from about 50% to about 65% w/w, or from about 55% to about 65% w/w ebselen. In further embodiments, the pharmaceutical composition comprises about 30% w/w, about 35% w/w, about 40% w/w, about 45% w/w, about 50% w/w, about 53% w/w, about 55% w/w, about 57% w/w, about 60% w/w, about 63% w/w, about 65% w/w, about 68% w/w, about 70% w/w, about 75% w/w, about 80% w/w ebselen. In a specific embodiment, the pharmaceutical composition comprises about 50% w/w ebselen. In a specific embodiment, the pharmaceutical composition comprises about 53% w/w ebselen. In a specific embodiment, the pharmaceutical composition comprises about 55% ebselen. In a specific embodiment, the pharmaceutical composition comprises about 57% w/w ebselen. In a specific embodiment, the pharmaceutical composition comprises about 60% w/w ebselen. In a specific embodiment, the pharmaceutical composition comprises about 65% w/w ebselen.

In some embodiments, the pharmaceutical composition comprises from 30% to 80% w/w ebselen. In further embodiments, the pharmaceutical composition comprises from 30% to 75% w/w, or from 30% to 70% w/w, 30% to 65% w/w, 30% to 60%, 40% to 60%, or from 50% to 65% w/w, or from 55% to 65% w/w ebselen. In further embodiments, the pharmaceutical composition comprises 30% w/w, 35% w/w, 40% w/w, 45% w/w, 50% w/w, 53% w/w, 55% w/w, 57% w/w, 60% w/w, 63% w/w, 65% w/w, 68% w/w, 70% w/w, 75% w/w, 80% w/w ebselen. In a specific embodiment, the pharmaceutical composition comprises 50% w/w ebselen. In a specific embodiment, the pharmaceutical composition comprises 53% w/w ebselen. In a specific embodiment, the pharmaceutical composition comprises 55% ebselen. In a specific embodiment, the pharmaceutical composition comprises 57% w/w ebselen. In a specific embodiment, the pharmaceutical composition comprises 60% w/w ebselen. In a specific embodiment, the pharmaceutical composition comprises 65% w/w ebselen.

### 4.1.2. Excipients

The pharmaceutical compositions provided in accordance with the present disclosure can be administered orally. In certain embodiments, the disclosure therefore provides pharmaceutical compositions that ebselen as described herein, and one or more pharmaceutically acceptable excipients or carriers including but not limited to, inert solid diluents and fillers, diluents, including sterile aqueous solution and various organic solvents, permeation enhancers, solubilizers, surfactants, disintegrants, lubricants, binders, glidants, adjuvants, and combinations thereof. Such compositions are prepared in a manner well known in the pharmaceutical art (see, e.g., Remington: The Science and Practice of Pharmacy (Remington: The Science and Practice of Pharmacy, 23rd Edition, ISBN-13: 978-0128200070); and Modern Pharmaceutics, Marcel Dekker, Inc., 4th Ed. (G. S. Banker & C. T. Rhodes, Eds.).

The pharmaceutical compositions may be administered in a fixed dose combination by oral administration. Administration may be via capsule, tablet, or the like. In one embodiment, the ebselen formulation is in the form of a tablet. In a further embodiment, the tablet is a compressed tablet. In making the pharmaceutical compositions that include the solid described herein, the active ingredient is usually diluted by an excipient and/or enclosed within such a carrier that can be in the form of a capsule, tablet, sachet, or other container. When the excipient serves as a diluent, it can be in the form of a solid, semi-solid or liquid material (as above), which acts as a vehicle, carrier or medium for the active ingredient.

The pharmaceutical composition may be formulated for immediate release or sustained release. A “sustained release formulation” is a formulation which is designed to slowly release a therapeutic agent in the body over an extended period of time, whereas an “immediate release formulation” is a formulation which is designed to quickly release a therapeutic agent in the body over a shortened period of time. In some cases, the immediate release formulation may be coated such that the therapeutic agent is only released once it reached the desired target in the body (e.g., the stomach). In a specific embodiment, the pharmaceutical composition is formulated for immediate release.

The pharmaceutical composition may further comprise pharmaceutical excipients such as fillers or diluents, binders, glidants, disintegrants, lubricants, solubilizers, and combinations thereof. Some examples of suitable excipients are described herein. When the pharmaceutical composition is formulated into a tablet, the tablet may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In some embodiments, the pharmaceutical composition comprises a filler, such as a carbohydrate or a protein filler. In some embodiments, the filler is selected from the group consisting of dicalcium phosphate, cellulose, microcrystalline cellulose, hydroxypropylmethyl cellulose, sodium carboxymethylcellulose, compressible sugars, dibasic calcium phosphate dehydrate, lactose, lactose monohydrate, sucrose, mannitol, sorbital, starch from corn, wheat, rice, potato or other plants, tribasic calcium phosphate, a gum (e.g., Arabic or tragacanth), proteins (e.g., gelatin or collagen) and combinations thereof.

In further embodiments, the pharmaceutical composition comprises one or more fillers in an amount from about 10 to about 65% w/w, or from about 10 to about 60% w/w, or from about 10% to about 55% w/w, or from about 10 to about 50% w/w, or from about 20 to about 50% w/w, or from about 25 to about 50% w/w, or from about 25 to about 45% w/w, or from about 25 to 40% w/w, or from about 30 to about 40%, or about 34 to about 40% w/w. In specific embodiments, one or more fillers is present in an amount of about 25%, or about 22%, or about 24%, or about 26%, or about 28%, or about 30%, or about 32%, or about 34%, or about 36%, or about 38%, or about 40 w/w. In a further specific embodiment, the filler is microcrystalline cellulose present in an amount of about 35% w/w. In a further specific embodiment, the filler is microcrystalline cellulose present in an amount of about 38% w/w.

In further embodiments, the pharmaceutical composition comprises one or more fillers in an amount from 10 to 65% w/w, or from 10 to 60% w/w, or from 10% to 55% w/w, or from 10 to 50% w/w, or from 20 to 50% w/w, or from 25 to 50% w/w, or from 25 to 45% w/w, or from 25 to 40% w/w, or from 30 to 40%, or 34 to 40% w/w. In specific embodiments, the one or more fillers is present in an amount of 25%, or 22%, or 24%, or 26%, or 28%, or 30%, or 32%, or 34%, or 36%, or 38%, or 40 w/w. In a further specific embodiment, the filler is microcrystalline cellulose present in an amount of 35% w/w. In a further specific embodiment, the filler is microcrystalline cellulose present in an amount of 38% w/w.

In some embodiments, the pharmaceutical composition comprises a disintegrant or solubilizing agent selected from cross-linked polyvinyl pyrrolidone, agar, alginic acid or a salt thereof (e.g., sodium alginate), croscarmellose sodium, crospovidone, modified corn starch, pregelatinized starch, sodium starch glycolate, and combinations thereof.

In certain embodiments, the pharmaceutical composition comprises one or more disintegrants in an amount from about 1 to about 10% w/w, or from about 1 to about 9% w/w, or from about 1 to about 8% w/w, or from about 2 to about 8% w/w, or from about 4 to about 8% w/w. In specific embodiments, one or more disintegrants is present in an amount of about 1%, or about 2%, or about 3%, or about 4%, or about 5%, or about 6%, or about 7%, or about 8%, or about 9%, or about 10% w/w. In a further specific embodiment, the disintegrant is croscarmellose sodium present in an amount of about 4% w/w. In a further specific embodiment, the disintegrant is croscarmellose sodium present in an amount of about 2% w/w. In a further specific embodiment, the disintegrant is croscarmellose sodium present in an amount of about 8% w/w.

In certain embodiments, the pharmaceutical composition comprises one or more disintegrants in an amount from about 1 to 10% w/w, or from 1 to 9% w/w, or from 1 to 8% w/w, or from 2 to 8% w/w, or from 4 to 8% w/w. In specific embodiments, one or more disintegrants is present in an amount of 1%, or 2%, or 3%, or 4%, or 5%, or 6%, or 7%, or 8%, or 9%, or 10% w/w. In a further specific embodiment, the disintegrant is croscarmellose sodium present in an of 4% w/w. In a further specific embodiment, the disintegrant is croscarmellose sodium present in an of 2% w/w. In a further specific embodiment, the disintegrant is croscarmellose sodium present in an of 8% w/w.

In some embodiments, the pharmaceutical composition comprises a lubricant selected from the group consisting of calcium stearate, magnesium stearate, polyethylene glycol, sodium stearyl fumarate, stearic acid, and combinations thereof.

In further embodiments, the pharmaceutical composition comprises one or more lubricants in an amount from about 0.1 to about 1% w/w, or from about 0.1 to about 0.7% w/w, or from about 0.25 to about 1% w/w, or from about 0.25 to about 0.7% w/w, or from about 0.25 to about 0.6% w/w, or from about 0.25 to about 0.5% w/w, or from about 0.3 to about 0.5% w/w. In specific embodiments, the lubricant is magnesium stearate present in an amount of about 0.1%, or about 0.2, or about 0.3%, or about 0.4%, or about 0.5% w/w. In a further specific embodiment, the lubricant is magnesium stearate present in an amount of about 0.5% w/w.

In further embodiments, the pharmaceutical composition comprises one or more lubricants in an amount from 0.1 to 1% w/w, or from 0.1 to 0.7% w/w, or from 0.25 to 1% w/w, or from 0.25 to 0.7% w/w, or from 0.25 to 0.6% w/w, or from 0.25 to 0.5% w/w, or from 0.3 to 0.5% w/w. In specific embodiments, the lubricant is magnesium stearate present in an amount of 0.1%, or 0.2, or 0.3%, or 0.4%, or 0.5% w/w. In a further specific embodiment, the lubricant is magnesium stearate present in an amount of 0.5% w/w.

In some embodiments, the pharmaceutical composition comprises a glidant selected from the group consisting of colloidal silicon dioxide, talc, and combinations thereof.

In further embodiments, the pharmaceutical composition comprises one or more glidants in an amount from about 0.1 to about 0.5% w/w, or from about 0.2 to about 0.5% w/w, or from about 0.3 to about 0.5% w/w. In specific embodiments, one or more glidants is present in an amount of about 0.1% w/w, 0.2% w/w, 0.3% w/w, 0.4% w/w, 0.5% w/w. In a further specific embodiment, the glidant is colloidal silicon dioxide present in an amount of about 0.5% w/w.

In further embodiments, the pharmaceutical composition comprises one or more glidants in an amount from 0.1 to 0.5% w/w, or from 0.2 to 0.5% w/w, or from 0.3 to 0.5% w/w. In specific embodiments, one or more glidants is present in an amount of 0.1% w/w, 0.2% w/w, 0.3% w/w, 0.4% w/w, 0.5% w/w. In a further specific embodiment, the glidant is colloidal silicon dioxide present in an amount of 0.5% w/w.

In one embodiment, the pharmaceutical composition comprises about 45 to about 60% w/w ebselen. In a related embodiment, the composition comprises about 50 to about 60% w/w ebselen. In some embodiments, the composition comprises about 57% w/w ebselen. In some embodiments, the composition further comprises a) about 25 to about 40% w/w filler, b) about 4 to about 8% w/w disintegrant, c) about 0.25 to about 0.5% w/w lubricant, and d) about 0.3 to about 0.5% w/w glidant. In some embodiments, the composition further comprises a) about 25 to about 40% w/w microcrystalline cellulose, b) about 4 to about 8% w/w croscarmellose sodium, c) about 0.25 to about 0.5% w/w magnesium stearate, and d) about 0.3 to about 0.5% w/w colloidal silicon dioxide. In some embodiments, the composition comprises a) about 57% w/w ebselen, b) about 34-40% w/w filler, c) about 4% w/w disintegrant, d) about 0.25-0.5% w/w lubricant, e) about 0.3-0.5% w/w glidant. In some embodiments, the composition comprises a) about 57% w/w ebselen, b) about 34-40% w/w microcrystalline cellulose, c) about 4% w/w croscarmellose sodium, d) about 0.25-0.5% w/w magnesium stearate, e) about 0.3-0.5% w/w colloidal silicon dioxide.

In one embodiment, the pharmaceutical composition comprises 45 to 60% w/w ebselen. In a related embodiment, the composition comprises 50 to 60% w/w ebselen. In some embodiments, the composition comprises 57% w/w ebselen. In some embodiments, the composition further comprises a) 25 to 40% w/w filler, b) 4 to 8% w/w disintegrant, c) 0.25 to 0.5% w/w lubricant, and d) 0.3 to 0.5% w/w glidant. In some embodiments, the composition further comprises a) 25 to 40% w/w microcrystalline cellulose, b) 4 to 8% w/w croscarmellose sodium, c) 0.25 to 0.5% w/w magnesium stearate, and d) 0.3 to 0.5% w/w colloidal silicon dioxide. In some embodiments, the composition comprises a) 57% w/w ebselen, b) 34-40% w/w filler, c) 4% w/w disintegrant, d) 0.25-0.5% w/w lubricant, e) 0.3-0.5% w/w glidant. In some embodiments, the composition comprises a) 57% w/w ebselen, b) 34-40% w/w microcrystalline cellulose, c) 4% w/w croscarmellose sodium, d) 0.25-0.5% w/w magnesium stearate, e) 0.3-0.5% w/w colloidal silicon dioxide.

In some embodiments the pharmaceutical composition comprises:

In some

| | Amount (% w/w) |
|---|---|
| ebselen | 45-60 |
| one or more fillers | 25-40 |
| one or more disintegrants | 2-8 |
| one or more lubricants | 0.1-1 |
| one or more glidants | 0.1-1 |

embodiments, the pharmaceutical composition comprises:

| | Amount (% w/w) |
|---|---|
| ebselen | 50-60 |
| one or more fillers | 34-40 |
| one or more disintegrants | 4-8 |
| one or more lubricants | 0.25-0.5 |
| one or more glidants | 0.3-0.5 |

In certain embodiments, the ebselen is present in an amount to 55-60% w/w, such as 55%, 56%, 57%, 58%, 59% or 60%.

The pharmaceutical compositions described herein can be formulated with ebselen as the sole pharmaceutically active ingredient in the composition or can be combined with other active ingredients (e.g., as described herein).

In certain embodiments, the pharmaceutical composition is formulated into one or more suitable pharmaceutical preparations, such as solutions, suspensions, powders, sustained release formulations or elixirs in sterile solutions or suspensions for parenteral administration, or as transdermal patch preparation and dry powder inhalers.

In compositions provided herein, ebselen described herein may be mixed with a suitable pharmaceutical carrier. The concentration of ebselen in the compositions can, for example, be effective for delivery of an amount, upon administration an enhanced maximum blood plasma concentration (Cₘₐₓ) is achieved for ebselen that is greater than achieved with the subject control ebselen formulation (e.g., as described herein), and is achieved within 4 hours or less of administration.

In certain embodiments, the pharmaceutical compositions provided herein are formulated for single dosage administration. To formulate a composition, the weight fraction of each active is dissolved, suspended, dispersed or otherwise mixed in a selected carrier at an effective concentration such that an enhanced maximum blood plasma concentration (Cₘₐₓ) is achieved for ebselen that is greater than achieved with the subject control ebselen formulation (e.g., as described herein), and is achieved within 4 hours or less of administration.

Concentrations of the ebselen in a pharmaceutical composition provided herein will depend on, e.g., the physicochemical characteristics of the compound, the dosage schedule, and amount administered as well as other factors known to those of skill in the art. For example, if the composition comprises a salt of ebselen the amount of said salt to be administered and/or to be incorporated into a pharmaceutical composition (i.e., pharmaceutical dosage form) needs to be adjusted to take account of the molecular weight difference between the free base and salt form. For instance, in expressing dose amounts in the label and/or product information of authorized medicinal products comprising a salt form of an active compound that can also be used in free base form, it is customary practice to specify the dose of the free base to which the dose of the salt as used is equivalent.

Pharmaceutical compositions described herein are provided for administration to a subject, for example, humans or animals (e.g., mammals) in unit dosage forms, such as sterile parenteral (e.g., intravenous) solutions or suspensions containing suitable quantities of the compounds or pharmaceutically acceptable derivatives thereof. Pharmaceutical compositions are also provided for administration to humans and animals in unit dosage form, including oral or nasal solutions or suspensions and oil-water emulsions containing suitable quantities of ebselen or pharmaceutically acceptable derivatives thereof. The ebselen is, in certain embodiments, formulated and administered in unit-dosage forms or multiple-dosage forms. Unit-dose forms as used herein refers to physically discrete units suitable for human or animal (e.g., mammal) subjects and packaged individually as is known in the art. Each unit-dose contains a predetermined quantity of ebselen sufficient to produce the desired therapeutic effect, in association with the required pharmaceutical carrier, vehicle or filler. Examples of unit-dose forms include ampoules and syringes and individually packaged tablets. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of capsules or bottles. Hence, in specific aspects, multiple dose form is a multiple of unit-doses which are not segregated in packaging.

In some embodiments the subject pharmaceutical composition is formulated as tablets, pills, dragees, capsules, liquids, gels, syrups, slurries, suspensions, and the like, suitable for ingestion by a subject. In certain cases, the pharmaceutical composition is formulated as a dragee, and dragee cores are provided with suitable coatings such as concentrated sugar solutions, which may also contain gum arabic, tale, polyvinylpyrrolidone, carbopol gel, polyethylene glycol, and/or titanium dioxide, lacquer solutions, and suitable organic solvents or solvent mixtures. Dyestuffs or pigments may be added to the dragee coatings for product identification or to characterize the quantity of active compound (i.e., dosage).

In some embodiments, the subject pharmaceutical composition is formulated for oral use as push-fit capsules made of gelatin, as well as soft, sealed capsules made of gelatin having a coating such as glycerol or sorbitol. Push-fit capsules can contain ebselen mixed excipients as described herein, e.g., fillers or binders such as lactose or starches, lubricants such as talc or magnesium stearate, and optionally stabilizers. In soft capsules, the ebselen may be dissolved or suspended in suitable liquids, such as fatty oils, liquid paraffin, or liquid polyethylene glycol with or without stabilizers.

In certain embodiments, the ebselen described herein are in a liquid pharmaceutical formulation. Liquid pharmaceutically administrable formulations can, for example, be prepared by dissolving, dispersing, or otherwise mixing the active compounds and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington: The Science and Practice of Pharmacy (Remington: The Science and Practice of Pharmacy, 23rd Edition, ISBN-13: 978-0128200070) Dosage forms or compositions containing ebselen in the ranges disclosed herein with the balance made up from non-toxic carrier can be prepared.

Parenteral administration, in certain embodiments, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. Other routes of administration may include, enteric administration, intracerebral administration, nasal administration, intraarterial administration, intracardiac administration, intraosseous infusion, intrathecal administration, and intraperitoneal administration.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Pharmaceutical carriers also include ethyl alcohol, polyethylene glycol and propylene glycol for water miscible vehicles; and sodium hydroxide, hydrochloric acid, citric acid or lactic acid for pH adjustment.

In certain embodiments, intravenous or intraarterial infusion of a sterile aqueous solution containing the combination described herein is an effective mode of administration.

Another embodiment is a sterile aqueous or oily solution or suspension containing a conjugate described herein injected as necessary to produce the desired pharmacological effect.

In certain embodiments, the pharmaceutical formulations are lyophilized powders, which can be reconstituted for administration as solutions, emulsions and other mixtures.

They can also be reconstituted and formulated as solids or gels.

The lyophilized powder is prepared by dissolving a compound provided herein, in a suitable solvent. In some embodiments, the lyophilized powder is sterile. Suitable solvents can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. A suitable solvent can also contain a buffer, such as citrate, sodium or potassium phosphate or other such buffer known to those of skill in the art at, in certain embodiments, about neutral pH. Subsequent sterile filtration of the solution followed by lyophilization under standard conditions known to those of skill in the art provides an example of a formulation. In certain embodiments, the resulting solution will be apportioned into vials for lyophilization. Lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier.

In certain embodiments, the pharmaceutical composition is formulated in as a solid dosage form, such as a tablet (e.g., as described herein below).

### 4.1.3. Additional Active Agents

The pharmaceutical compositions disclosed herein may optionally include one or more additional active agents. In some embodiments, at least one of the one or more additional active agents is an otoprotectant agent. In some embodiments, at least one of the one or more additional active agents is N-acetylcysteine or allopurinol.

Some otoprotectant agents that can be included in the subject pharmaceutical compositions include glutathione and glutathione precursors. Examples of otoprotectant agents in this category include but are not limited to, methionine, N-acetyl-DL-methionine, S-adenosylmethionine, cysteine, homocysteine, N-acetylcysteine, glutathione, glutathione ethylester, glutathione diethylester, glutathione triethylester, cysteamine, cystathione, N,N′-diacetyl-L-cystine (DiNAC), 2(R,S)-D-ribo-(1′,2′,3′,4′-tetrahydroxybutyl)-thiazolidine-4(R)-carboxylic acid (RibCys), 2-alkylthiazolidine 2(R,S)-D-ribo-(1′,2′,3′,4′-tetrahydroxybutyl)thiazolidine (RibCyst), and 2-oxo-L-thiazolidine-4-carboxylic acid (OTCA).

Xanthine oxidase inhibitors, for example allopurinol and its tautomers, can also be included in the subject pharmaceutical compositions. Examples of allopurinol derivatives include but are not limited to, 1-methylallopurinol, 2-methylallopurinol, 7-methylallopurinol, 1,5-dimethylallopurinol, 2,5-dimethylallopurinol, 1,7-dimethylallopurinol, 2,7 dimethylallopurinol, 5,7-dimethylallopurinol, 2,5,7-trimethylallopurinol, 1-ethoxycarbonylallopurinol, and 1-ethoxycarbonyl-5-methylallopurinol.

Additional glutathione peroxidase mimics can also be included in the subject pharmaceutical compositions. Examples of additional glutathione peroxidase mimics include but are not limited to, 6A,6B-diseleninic acid-6A′,6B′-selenium bridged-cyclodextrin (6-diSeCD), and 2,2′-diseleno-bis-Beta-cyclodextrin (2-diSeCD).

In some embodiments, the combination of ebselen, and one or more additional active agents according to the present disclosure can allow for a reduction in the dose of ebselen.

A dose reduction can be beneficial for patients which otherwise would potentially suffer from side effects in a therapy using a higher dose of ebselen. As such, the pharmaceutical composition and methods according to the present disclosure can exhibit less side effects to a corresponding monotherapy with ebselen, thereby making the therapy more tolerable and improving an individual's compliance with the treatment.

In certain embodiments, the pharmaceutical composition comprises from 1 to 50% w/w of an additional active agent, such as 1 to 45% w/w, 1 to 40% w/w, 1 to 35% w/w, 1 to 30% w/w, 1 to 25% w/w, 1 to 20% w/w, 1 to 15% w/w, 1 to 10% w/w, or 1 to 5% w/w of an additional active agent.

### 4.2. Pharmaceutical Dosage Forms

As summarized above, this disclosure provides a pharmaceutical dosage form comprising the pharmaceutical composition described herein. The disclosure provides for tablets, pills, capsules and the like, comprising the pharmaceutical compositions or dosage forms described herein. Accordingly, in some embodiments, the pharmaceutical dosage from is a solid dosage form. In certain cases, the solid dosage form is a capsule. In certain cases, the capsule is an opaque hard gelatin capsule. In certain cases, the dosage form is a tablet. In certain cases, the tablet is an uncoated pressed tablet.

The tablets or pills of the present disclosure may be coated to provide a dosage form affording the advantage of prolonged action or to protect from the acid conditions of the stomach. In certain cases, the tablets or pills of the present disclosure are uncoated. The tablets may also be formulated for immediate release. In certain embodiments, the tablet comprises a film coating. A film coating may be useful for limiting photolytic degradation. Suitable film coatings are selected by routine screening of commercially available preparations. In one embodiment, the film coating is a hypromellose-based coating. In certain embodiments, the coating comprises a film-forming agent, a plasticizer, a glidant and optionally one or more pigments. An exemplary film coating composition may comprise hydroxypropyl methylcellulose (HPMC), lactose monohydrate, titanium dioxide, and triglyceride 1,2,3-triacetoxypropane (triacetin). In certain cases, the film coating composition may comprise hydroxypropyl methylcellulose (HPMC), polyethylene glycol (PEG), talc, titanium dioxide and optionally iron oxide, including iron oxide red and/or yellow.

The tablets may be formulated into a monolayer or bilayer tablet. Typically, monolayer tablets comprise the active ingredients (i.e., ebselen and optionally co-mixed with one or more other actives) in a single uniform layer. For making monolayer tablets, exemplary methods include dry granulation, direct compression, and wet granulation. The direct compression tablet process uses two primary process steps: blending the active ingredients with the excipients and compressing the finished tablet.

In some embodiments, the present disclosure provides a dry granulation process for making the subject pharmaceutical dosage form, wherein said process comprises the steps of:
(1) mixing the active ingredient with either all or a portion of the excipients in a mixer;
(2) compaction of the mixture of step (1) on a suitable roller compactor;
(3) reducing the ribbons obtained during step (2) to granules, preferably small granules, by suitable milling or sieving steps;
(4) optionally mixing the granules of step (3) with the remaining excipients in a mixer to obtain the final mixture;
(5) tableting the granules of step (3) or the final mixture of step (4) by compressing it on a suitable tablet press to produce the tablet cores;
(6) optionally film-coating of the tablet cores of step (5) with a non-functional coat.

In certain embodiments, the present disclosure provides a pharmaceutical dosage form (e.g., as described herein) obtainable by a dry granulation process.

In some embodiments, the present disclosure provides a wet granulation process for making the subject pharmaceutical dosage form, wherein said process comprises the steps of:
(1) Premixing the active ingredient and the main portion of the excipients including the binder in a mixer to obtain a pre-mixture;
(2) granulating the pre-mixture of step (1) by adding the granulation liquid, e.g., purified water;
(3) drying the granules of step (2) in a fluidized bed dryer or a drying oven;
(4) optionally dry sieving of the dried granules of step (3);
(5) mixing the dried granules of step (4) with the remaining excipients like glidant and lubricant in a mixer to obtain the final mixture;
(6) tableting the final mixture of step (5) by compressing it on a suitable tablet press to produce tablets cores;
(7) optionally film-coating of the tablet cores of step (6) with a non-functional coat.

In certain embodiments, the present disclosure provides a pharmaceutical dosage form (e.g., as described herein) obtainable by a wet granulation process.

In some embodiments, the present disclosure provides a direct compression process for making the subject pharmaceutical dosage form, wherein said process comprises the steps of:
(1) Premixing the active ingredient and the main portion of the excipients in a mixer to obtain a pre-mixture;
(2) optionally dry screening the pre-mixture through a screen in order to segregate cohesive particles and to improve content uniformity;
(3) mixing the pre-mixture of step (1) or (2) in a mixer, optionally by adding remaining excipients to the mixture and continuing mixing;
(4) tableting the final mixture of step (3) by compressing it on a suitable tablet press to produce the tablet cores;
(5) optionally film-coating of the tablet cores of step (4) with a non-functional coat.

In certain embodiments, the present disclosure provides a pharmaceutical dosage form obtainable (e.g., as described herein) by a direct compression process.

In certain embodiments, the pharmaceutical dosage form comprises the ebselen in a therapeutically effective amount (e.g., as described herein for ebselen).

In certain embodiments, the pharmaceutical dosage form comprises from about 45% to about 60% w/w ebselen. In some embodiments, the pharmaceutical dosage form comprises from about 50% to about 60% w/w ebselen. In some embodiments, the pharmaceutical dosage form comprises about 50% w/w, about 51% w/w, about 52% w/w, about 53% w/w, about 54% w/w, about 55% w/w, about 56% w/w, about 57% w/w, about 58% w/w, about 59% w/w, or about 60% w/w ebselen. In a specific embodiment, the pharmaceutical dosage form comprises about 55-58% w/w ebselen.

In certain embodiments, the pharmaceutical dosage form comprises from 45% to 60% w/w ebselen. In some embodiments, the pharmaceutical dosage form comprises from 50% to 60% w/w ebselen. In some embodiments, the pharmaceutical dosage form comprises 50% w/w, 51% w/w, 52% w/w, 53% w/w, 54% w/w, 55% w/w, 56% w/w, 57% w/w, 58% w/w, 59% w/w, or 60% w/w ebselen. In a specific embodiment, the pharmaceutical dosage form comprises 55-58% w/w ebselen.

In some embodiments, the pharmaceutical dosage form comprises from about 200 mg to about 600 mg ebselen. In some embodiments, the pharmaceutical dosage form comprises from about 200 mg to about 250 mg ebselen, or from about 200 mg to about 300 mg ebselen, or from about 200 mg to about 350 mg ebselen, or from about 300 mg to about 350 mg ebselen, or from about 350 mg to about 400 mg ebselen, or from 350 to about 450 ebselen, or from about 400 to about 450 ebselen, or from about 400 to about 500 mg ebselen, or from about 400 to about 550 mg ebselen, or from about 400 to about 600 mg ebselen. In further embodiments, the pharmaceutical dosage form comprises about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, or about 600 mg ebselen. In a specific embodiment, the pharmaceutical dosage form comprises about 400 mg ebselen. In a specific embodiment, the pharmaceutical dosage form comprises about 200 mg ebselen. In another embodiment, the pharmaceutical dosage form comprises about 600 mg ebselen.

In some embodiments, the pharmaceutical dosage form comprises from 200 mg to 600 mg ebselen. In some embodiments, the pharmaceutical dosage form comprises from 200 mg to 250 mg ebselen, or from 200 mg to 300 mg ebselen, or from 200 mg to 350 mg ebselen, or from 300 mg to 350 mg ebselen, or from 350 mg to 400 mg ebselen, or from 350 to 450 ebselen, or from 400 to 450 ebselen, or from 400 to 500 mg ebselen, or from 400 to 550 mg ebselen, or from 400 to 600 mg ebselen. In further embodiments, the pharmaceutical dosage form comprises 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, or 600 mg ebselen. In a specific embodiment, the pharmaceutical dosage form comprises 400 mg ebselen. In a specific embodiment, the pharmaceutical dosage form comprises 200 mg ebselen. In another embodiment, the pharmaceutical dosage form comprises 600 mg ebselen.

In certain embodiments, the pharmaceutical dosage form comprises one or more excipients (e.g., as described herein). In certain embodiments, the pharmaceutical dosage form comprises one or more fillers. In certain embodiments, the pharmaceutical dosage from comprises microcrystalline cellulose.

In certain embodiments, the pharmaceutical dosage form comprises microcrystalline cellulose in an amount from about 140 mg to 280 mg, such as 145 mg to 275 mg, 145 mg to 270 mg, 145 mg to 265 mg, or 150 to 265 mg. In a specific embodiment, the microcrystalline cellulose is in an amount of about 265 mg.

In certain embodiments, the pharmaceutical dosage form comprises microcrystalline cellulose in an amount from 140 mg to 280 mg, such as 145 mg to 275 mg, 145 mg to 270 mg, 145 mg to 265 mg, or 150 to 265 mg. In a specific embodiment, the microcrystalline cellulose is in an amount of 265 mg.

In certain embodiments, the pharmaceutical dosage form comprises one or more disintegrants. In certain cases, the disintegrant is croscarmellose sodium. In certain embodiments, the pharmaceutical dosage form comprises croscarmellose sodium in an amount from about 14 mg to 65 mg, such as about 14 mg to 60 mg, 14 mg to 50 mg, 14 mg to 45 mg, 14 mg to 40 mg, 14 to 35 mg, or about 14 to 30 mg. In certain embodiments, the pharmaceutical dosage from comprises croscarmellose sodium in an amount of from about 20 mg to 40 mg, such as about 20 mg to 35 mg, about 20 mg to 30 mg, or about 25 mg to 30 mg.

In a specific embodiment, the croscarmellose sodium is in an amount of about 28 mg.

In certain embodiments, the pharmaceutical dosage form comprises croscarmellose sodium in an amount from 14 mg to 65 mg, such as 14 mg to 60 mg, 14 mg to 50 mg, 14 mg to 45 mg, 14 mg to 40 mg, 14 to 35 mg, or 14 to 30 mg. In certain embodiments, the pharmaceutical dosage from comprises croscarmellose sodium in an amount of from 20 mg to 40 mg, such as 20 mg to 35 mg, 20 mg to 30 mg, or 25 mg to 30 mg. In a specific embodiment, the croscarmellose sodium is in an amount of 28 mg.

In certain embodiments, the pharmaceutical dosage form comprises one or more lubricants. In certain cases, the lubricant is magnesium stearate. In certain embodiments, the pharmaceutical dosage form comprises magnesium stearate in an amount from about 0.7 mg to 7 mg, such as about 0.7 mg to 6 mg, about 0.7 mg to 5 mg, about 0.7 mg to 4 mg, or about 0.7 to 3.5 mg. In certain embodiments, the pharmaceutical dosage from comprises magnesium stearate in an amount from about 1.5 mg to 5 mg, such as about 1.5 to 4 mg, about 2 mg to 4 mg, about 2.5 mg to 4 mg, or about 3 mg to 4 mg. In a specific embodiment, the magnesium stearate is in an amount of about 3.5 mg.

In certain embodiments, the pharmaceutical dosage form comprises magnesium stearate in an amount from 0.7 mg to 7 mg, such as 0.7 mg to 6 mg, 0.7 mg to 5 mg, 0.7 mg to 4 mg, or 0.7 to 3.5 mg. In certain embodiments, the pharmaceutical dosage from comprises magnesium stearate in an amount from 1.5 mg to 5 mg, such as 1.5 to 4 mg, 2 mg to 4 mg, 2.5 mg to 4 mg, or 3 mg to 4 mg. In a specific embodiment, the magnesium stearate is in an amount of 3.5 mg.

In certain embodiments, the pharmaceutical dosage form comprises one or more glidants. In certain cases, the glidant is colloidal silicon dioxide. In certain embodiments, the pharmaceutical dosage form comprises colloidal silicon dioxide in an amount from about 0.7 mg to 7 mg, such as about 0.7 mg to 6 mg, 0.7 mg to 5 mg, 0.7 mg to 4 mg, or about 0.7 to 3.5 mg. In certain embodiments, the pharmaceutical dosage from comprises colloidal silicon dioxide in an amount from about 1.5 mg to 5 mg, such as about 1.5 to 4 mg, about 2 mg to 4 mg, about 2.5 mg to 4 mg, or about 3 mg to 4 mg. In a specific embodiment, the colloidal silicon dioxide is in an amount of about 3.5 mg.

In certain embodiments, the pharmaceutical dosage form comprises colloidal silicon dioxide in an amount from 0.7 mg to 7 mg, such as 0.7 mg to 6 mg, 0.7 mg to 5 mg, 0.7 mg to 4 mg, or 0.7 to 3.5 mg. In certain embodiments, the pharmaceutical dosage form comprises colloidal silicon dioxide in an amount from 1.5 mg to 5 mg, such as 1.5 to 4 mg, 2 to 4 mg, 2.5 mg to 4 mg, or 3 mg to 4 mg. In a specific embodiment, the colloidal silicon dioxide is in an amount of 3.5 mg.

In certain embodiments, the pharmaceutical dosage from comprises a) 200-400 mg ebselen; b) 140-280 mg microcrystalline cellulose; c) 14 to 63 mg croscarmellose sodium; d) 0.7-7 mg magnesium stearate; and 0.7-7 mg colloidal silicon dioxide.

In certain embodiments, the pharmaceutical dosage from comprises a) 400 mg ebselen; b) 265 mg microcrystalline cellulose; c) 28 mg croscarmellose sodium; d) 3.5 mg magnesium stearate; and 3.5 mg colloidal silicon dioxide.

In certain embodiments, the pharmaceutical dosage form is a solid dosage form (e.g., a tablet, such as an uncoated pressed tablet) comprising an intra-granular phase and an extra-granular phase. In certain cases, the intra-granular phase includes ebselen and one or more excipients (e.g., as described herein), and the extra-granular phase includes additional excipients (e.g., lubricant and glidant). In certain embodiments, the intra-granular phase includes ebselen, filler, disintegrant and lubricant; and the extra-granular phase includes additional lubricant and a glidant.

In some embodiments the pharmaceutical dosage from comprises:

| | Amount (% w/w) |
|---|---|
| Intra-granular phase | |
| ebselen | 45-60 |
| one or more fillers | 25-40 |
| one or more disintegrants | 2-8 |
| one or more lubricants | 0.1-1 |
| Extra-granular phase | |
| One or more lubricants | 0.1-1 |
| one or more glidants | 0.1-1 |

where the fillers, disintegrants, lubricants and glidants are as described herein.

In certain embodiments, the pharmaceutical dosage form comprises: where the

| | Amount (% w/w) |
|---|---|
| Intra-granular phase | |
| ebselen | 50-60 |
| one or more fillers | 34-40 |
| one or more disintegrants | 4-8 |
| one or more lubricants | 0.25-0.5 |
| Extra-granular phase | |
| one or more lubricants | 0.25-0.5 |
| one or more glidants | 0.3-.05 |

fillers, disintegrants, lubricants and glidants are as described herein.

In some embodiments, the pharmaceutical dosage from comprises:

| | (Amount mg) |
|---|---|
| Intra-granular phase | |
| ebselen | 200-400 |
| one or more fillers | 140-280 |
| one or more disintegrants | 14-63 |
| one or more lubricants | 0.7-7 |
| Extra-granular phase | |
| one or more lubricants | 0.7-7 |
| one or more glidants | 0.7-7 |

where the fillers, disintegrants, lubricants and glidants are as described herein.

In some embodiments, the pharmaceutical dosage from comprises:

| | (Amount mg) |
|---|---|
| Intra-granular phase | |
| ebselen | 400 |
| microcrystalline cellulose | 265 |
| croscarmellose sodium | 28 |
| magnesium stearate | 1.75 |
| Extra-granular phase | |
| magnesium stearate | 1.75 |
| colloidal silicon dioxide | 3.5 |

In some embodiments, the present disclosure provides process for making a tablet comprising an intra-granular phase and an extra-granular phase, wherein said process comprises the steps of:
(1) mixing the active ingredient with either all or a portion of the excipients in a mixer to form an intra-granular phase;
(2) mixing suitable excipients to form an extra-granular phase;
(3) mixing the intra-granular phase of step (1) with the extra-granular phase formed in step (2);
(4) tableting the granules of step (3) by compressing it on a suitable tablet press to produce the tablet cores; and
(5) optionally film-coating of the tablet cores of step (4) with a non-functional coat.

In certain embodiments, the pharmaceutical dosage form is a bilayer tablets comprising two or more active ingredients (i.e., ebselen and one other, e.g., as described herein) in separate layers and can be made by making a blend comprising excipients and one active ingredient (i.e., ebselen), and making a separate blend comprising the additional active ingredient (e.g., as described herein) and excipients. One blend may then be pre-compressed, and the second blend may then be added on top of the first pre-compressed blends. The resulting tablet comprises two separate layers, each layer comprising a different active ingredient.

In some embodiments, the pharmaceutical dosage form (e.g., as described herein above) has a disintegration time of two hours or less and a dissolution rate of 70% or more at 30 minutes. In certain cases, the disintegration time is less than 2 hours, such as 1.75 hours or less, 1.5 hours or less, 1 hour or less, 0.5 hours or less, or even less. In certain cases, the pharmaceutical dosage form has a dissolution rate of more than 70% at 30 minutes, such as more than 75%, more than 80%, more than 85%, more than 90%, or more than 95% at 30 minutes.

In some embodiments, the subject pharmaceutical dosage form (e.g., as described herein) has a disintegration time of one hour or less and a dissolution rate of 80% or more at 30 minutes. In certain cases, the disintegration time is less than 1 hour, such as 46 minutes or less, 30 minutes or less, 15 minutes or less, 10 minutes or less, or even less. In certain cases, the pharmaceutical dosage form has a dissolution rate of more than 80% at 30 minutes, such as more than 85%, more than 90%, or more than 95% at 30 minutes.

In some embodiments, the subject pharmaceutical dosage form (e.g., as described herein) may be administered orally. In certain cases, the pharmaceutical dosage form (e.g., as described herein) is administered to a human subject to produce: a maximum blood plasma concentration (average Cₘₐₓ) that is greater than that achieved with a control ebselen formulation (e.g., as described herein); and/or a maximum blood plasma concentration (average Cₘₐₓ) that is achieved within 4 hours or less of administration.

In some embodiments, the solid dosage form is an uncoated pressed tablet. Accordingly, in some embodiments, there is provided an uncoated pressed tablet comprising: 400 mg ebselen, 265 mg microcrystalline cellulose, 28 mg croscarmellose sodium, 3.5 mg magnesium stearate, and 3.5 mg colloidal silicon dioxide.

In some embodiments, the uncoated pressed tablet comprises: an intra-granular phase comprising 400 mg ebselen, 265 mg microcrystalline cellulose, 28 mg croscarmellose sodium, and 1.75 mg magnesium stearate; and an extra-granular phase comprises 1.75 mg magnesium stearate and 3.5 mg colloidal silicon dioxide.

In some embodiments, the uncoated pressed tablet is formed at a pressure of from 1.5 to 4 MPa, such as 2 to 3.5 MPa or 2.5 to 3 MPa. In some embodiments, the uncoated pressed tablet is formed at a pressure of 3 MPa.

In some embodiments, the uncoated pressed tablet has a hardness of from 15 to 100N, such as 15 to 90N, 15 to 80N, 15 to 70N, 15 to 60N, 25 to 55N, 30 to 55N, 35 to 55N, 40 to 55N or 45 to 55N. In some embodiments, the uncoated pressed tablet has a harness of 50N.

In some embodiments, the subject uncoated pressed tablet has a disintegration time of two hours or less and a dissolution rate of 70% or more at 30 minutes. In certain cases, the disintegration time is less than 2 hours, such as 1.75 hours or less, 1.5 hours or less, 1 hour or less, 0.5 hours or less, or even less. In certain cases, the uncoated pressed tablet has a dissolution rate of more than 70% at 30 minutes, such as more than 75%, more than 80%, more than 85%, more than 90%, or more than 95% at 30 minutes.

In some embodiments, the subject uncoated pressed tablet has a disintegration time of one hour or less and a dissolution rate of 80% or more at 30 minutes. In certain cases, the disintegration time is less than 1 hour, such as 46 minutes or less, 30 minutes or less, 15 minutes or less, 10 minutes or less, or even less. In certain cases, the uncoated pressed table has a dissolution rate of more than 80% at 30 minutes, such as more than 85%, more than 90%, or more than 95% at 30 minutes.

In some embodiments, the subject uncoated pressed tablet has a dissolution profile that is reproducible. As used herein the term “reproducible dissolution profile” means that the difference between the dissolution profile of a first batch or lot measured over two hours and the dissolution profile of other lots or batches of the uncoated pressed tablet measured over two hours is not more than 5%, such as not more than 4%, not more than 3%, not more than 2%, or not more than 1%.

In some embodiments, the pharmaceutical composition, pharmaceutical dosage form, or tablet as described herein is free of negative drug-drug interactions. In a related embodiment, the pharmaceutical composition, pharmaceutical dosage form, or tablet is free of negative drug-drug interactions with other active agents. In a further embodiment, the pharmaceutical composition, pharmaceutical dosage form, or tablet as described herein is administrable without regard to food and with or without regard to the patient being on another therapeutic agent.

### 4.3. Methods of Use

As summarized above, also provided herein are methods of delivering the subject ebselen pharmaceutical dosage forms to a subject to achieve an enhanced maximum blood plasma concentration (Cₘₐₓ) with respect to a control ebselen formulation, and a maximum blood plasma concentration (Cₘₐₓ) for ebselen within 4 hours or less of administration.

An exemplary dosage may be a solid dosage form (e.g., a tablet, such as an uncoated pressed tablet) taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release. In certain embodiments, the pharmaceutical dosage form is administered twice daily. In some embodiments, the pharmaceutical dosage form is administered three times daily.

In some embodiments, multiple doses of the pharmaceutical dosage form are administered. The frequency of administration of the dosage form can vary depending on any of a variety of factors, e.g., severity of the symptoms, etc. For example, in some embodiments, the subject dosage form is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid). In certain embodiments, the subject dosage form is administered twice a day (bid).

The duration of administration of a pharmaceutical dosage from, e.g., the period of time over which ebselen is administered, can vary, depending on any of a variety of factors, e.g. patient response, etc. For example, an active agent can be administered over a period of time ranging from about one day to about one week, from about two weeks to about four weeks, from about one month to about two months, or from about two months to about four months, or more.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

Although the dosage used will vary depending on the clinical goals to be achieved, a suitable dosage range is in some embodiments one which provides a Cₘₐₓ of about 100 ng/mL to about 2500 ng/mL of ebselen in a blood sample taken from the individual being treated, within about 4 hours after administration of the ebselen to the individual.

In some embodiments, the subject pharmaceutical dosage form is administered in an amount that provides, a mean blood or plasma Cₘₐₓ of ebselen or at least 100 ng/mL, 150 ng/mL, 200 ng/mL, 250 ng/mL, 300 ng/mL, 350 ng/mL, 400 ng/mL, 450 ng/mL, 500 ng/mL, 550 ng/mL, 600 ng/mL, 650 ng/mL, 700 ng/mL, 750 ng/mL, 800 ng/mL, 850 ng/mL, 900 ng/mL, 950 ng/mL, 1000 ng/mL, 1050 ng/mL, 1100 ng/mL, 1150 ng/mL, 1200 ng/mL, 1250 ng/mL, 1300 ng/mL, 1350 ng/mL, 1400 ng/mL, 1450 ng/mL, 1500 ng/mL, 1550 ng/mL, 1600 ng/mL, 1650 ng/mL, 1700 ng/mL, 1750 ng/mL, 1800 ng/mL, 1850 ng/mL, 1900 ng/mL, 1950 ng/mL, 2000 ng/mL, 2250 ng/mL, or at least 2500 ng/mL.

In some embodiments, the subject pharmaceutical dosage form is administered in an amount that provides, within 1 day following administration, a mean blood or plasma Cₘₐₓ of ebselen that is at least 1.2-fold greater than the Cₘₐₓ achieved with the subject control ebselen formulation (e.g., as described herein in Example 1). In some cases, the maximum blood plasma concentration (Cₘₐₓ) achieved after one (1) day is at least about 1.5-fold greater than the Cₘₐₓ achieved with a control ebselen formulation, e.g., at least about 1.6-fold, 1.7-fold, 1.8-fold, 1.9-fold, 2.0-fold, 2.1-fold, 2.2-fold, 2.3-fold, 2.4-fold or 2.5-fold greater than the Cₘₐₓ achieved with a control ebselen formulation.

In some embodiments, the subject pharmaceutical dosage form is orally administered as a single tablet of 400 mg ebselen and exhibits greater (e.g., 1.2-fold greater or more) mean peak concentration (Cₘₐₓ) as compared to a dosage form administered as two capsules each comprising 200 mg ebselen, 120 mg microcrystalline cellulose, 28 mg croscarmellose sodium and 1.8 mg magnesium stearate. In certain cases, the orally administered single table of 400 mg ebselen exhibits at least a 1.2-fold, a 1.3-fold, a 1.4-fold, or at least a 1.5-fold greater mean peak concentration (Cₘₐₓ) as compared to a dosage form administered as two capsules each comprising 200 mg ebselen, 120 mg microcrystalline cellulose, 28 mg croscarmellose sodium and 1.8 mg magnesium stearate.

Depending on the subject and condition being treated and on the administration route, the pharmaceutical dosage form may be administered with ebselen dosages of, for example, 0.5 to 150 mg/kg body weight per day, e.g., from about 0.5 mg/kg body weight per day to about 1 mg/kg body weight per day, from about 1 mg/kg body weight per day to about 25 mg/kg body weight per day, from about 25 mg/kg body weight per day to about 50 mg/kg body weight per day, from about 50 mg/kg body weight per day to about 100 mg/kg body weight per day, from about 100 mg/kg body weight per day to about 125 mg/kg body weight per day, from about 125 mg/kg body weight per day to about 150 mg/kg body weight per day. The range is broad, since in general the efficacy of a therapeutic effect of ebselen for different mammals varies widely with doses typically being 20, 30 or even 40 times smaller (per unit body weight) in man than in the rat. Similarly, the mode of administration can have a large effect on dosage. Thus, for example, oral dosages may be about ten times the injection dose. Higher doses may be used for localized routes of delivery.

Depending on the subject and condition being treated and on the administration route, the pharmaceutical dosage form may be administered with ebselen dosages of, for example, 100 to 10,000 mg/dose, e.g., from about 100 mg/dose to about 150 mg/dose, from about 150 mg/dose to about 200 mg/dose, from about 200 mg/dose to about 250 mg/dose, from about 300 mg/dose to about 350 mg/dose, from about 350 mg/dose to about 400 mg/dose, from about 400 mg/dose to about 450 mg/dose, from about 450 mg/dose to about 500 mg/dose, form about 500 mg/dose to about 550 mg/dose, from about 600 mg/dose to about 650 mg/dose, from about 650 mg/dose to about 700 mg/dose, from about 700 mg/dose to about 750 mg/dose, from about 750 mg/dose to about 800 mg/dose, from about 800 mg/dose to about 850 mg/dose, from about 850 mg/dose to about 900 mg/dose, from about 900 mg/dose to about 950 mg/dose, from about 950 mg/dose to about 1000 mg/dose, from about 1,000 mg/dose to about 2,500 mg/dose, from about 2,500 mg/dose to about 5,000 mg/dose, from about 5,000 mg/dose to about 7500 mg/dose, from about 7,500 mg/dose to about 10,000 mg/dose. In some embodiments, the pharmaceutical dosage form may be administered in dosages of, for example, 0.1 to 10 g/dose, e.g., from about 0.1 g/dose to 0.25 g/dose, from about 0.2 g/dose to 0.4 g/dose, from about 0.4 g/dose to 0.5 g/dose, from about 0.5 g/dose to 1 g/dose, from about 1 g/dose to 3 g/dose, from about 3 g/dose to 5 g/dose, from about 5 g/dose to 6 g/dose, from about 6 g/dose to about 8 g/dose, from about 8 g/dose to about 10 g/dose.

In some embodiments, the pharmaceutical dosage form comprises 400 mg ebselen, and is administered orally (e.g., in a tablet) twice daily.

In some embodiments, the pharmaceutical dosage form is an uncoated pressed tablet comprising 400 mg ebselen, and is administered orally twice daily.

In some embodiments, the pharmaceutical dosage form comprises 800 mg ebselen, and is administered orally (e.g., in a tablet) twice daily.

### 4.3.1. Therapeutic Indications

Aspects of the present disclosure include methods of treating therapeutic indications of interest using the subject pharmaceutical compositions or dosage forms comprising ebselen (e.g., as described herein). The term “therapeutic indication” refers to any symptom, condition, disorder, or disease that may be alleviated, stabilized, improved, cured, or otherwise addressed by some form of treatment or other therapeutic intervention with ebselen. In some embodiments, methods of the present disclosure may include treating ebselen-related indications by administering compositions or pharmaceutical dosage forms disclosed herein (e.g., dosage forms comprising ebselen).

Ebselen has been shown to have use in the treatment and/or prevention of a range of diseases and conditions. Examples include but are not limited to, noise-induced hearing loss, Meniere's disease, chemotherapy induced ototoxicity, aminoglycoside-induced ototoxicity in patients with cystic fibrosis, and COVID-19.

### 4.4. Kits

Also provided herein is a pharmaceutical kit comprising a package containing a plurality of unit pharmaceutical dosage forms (e.g., as described herein) and instructions for use. In some embodiments, there is provided a pharmaceutical kit comprising a package containing a plurality of uncoated pressed tablets (e.g., as described herein) and instructions for use.

In accordance with embodiments of the invention, the pharmaceutical kit comprises a container, such as a high-density polyethylene (HDPE) bottles, or a box including one or more blister packs, wherein the bottles or blister packs can contain a plurality of solid unit pharmaceutical dosage forms as described herein. In certain embodiments, the container or pack comprises at least 5, at least 8, at least 10, at least 12 of at least 15 of said unit pharmaceutical dosage forms, e.g., 4, 5, 6, 7, 8, 9, 10, 11, 12, 13, 14, 15, 16, 17, 18, 19 or 20 of said unit dosage forms.

In accordance with the invention, the pharmaceutical kit comprises instructions (e.g., a leaflet) inserted into the container or box, typically a patient information leaflet containing printed information, which information may include a description of the form and composition of the unit pharmaceutical dosage forms contained in the kit, an indication of the therapeutic indications for which the product is intended, instructions as to how the product is to be used and information and warnings concerning adverse effects and contraindications associated with the use. In accordance with the present disclosure, the leaflet will usually contain the information concerning the therapeutic indications, uses, treatment regimens, etc. as described herein in relation to the methods of treatment of the present invention. In certain cases, the leaflet contains printed instructions to repeatedly (self-)administer the pharmaceutical unit dosage forms in order to treat and/or prevent a disease or condition, such as Meniere's Disease, conditions related to noise- or ototoxin-induced injury, acute lung infections and related conditions or diseases, such as coronavirus-mediated lung injuries.

### 4.5. Definitions

As used herein, the term “otoprotectant” refers to a chemical substance that is capable of ameliorating hearing loss.

As used herein, the term “ameliorating hearing loss” includes: (a) reducing the magnitude and/or duration of hearing loss; and/or (b) slowing the progression of hearing loss; and/or (c) preventing the onset of hearing loss that would occur without administration of an otoprotectant composition of the invention.

As used herein, the term “ototoxic agent” means an agent that is likely to impair the function of any component of the ear involved in hearing and, therefore, is likely to induce temporary or permanent hearing loss. Examples of ototoxic agents are ototoxic drugs and ototoxic noise.

As used herein, the term “exposure to an ototoxic agent” includes single or multiple exposures to an ototoxic agent that is recognized in the art as being likely to cause temporary or permanent hearing loss. For example, the Occupational Safety and Health Administration (OSHA) considers exposures to noise greater than or equal to 85 decibels (dB) to be hazardous to hearing. Thus, OSHA mandates that workers not be exposed to greater than or equal to 85 dB of noise over a continuous eight hour period based on a time weighted average, unless noise reduction measures (i.e., earmuffs) are employed.

As used herein, the term “otoprotectant composition” refers to a composition that includes at least one otoprotectant, and may include more than one otoprotectant.

Otoprotectant compositions may also include, in addition to one or more otoprotectant(s), pharmaceutically acceptable carriers that facilitate administration of an otoprotectant composition to a mammalian subject.

The abbreviation “mg” means milligrams.

The abbreviation “BID” means “twice daily.”

The abbreviation “QD” means “once daily.”

The terms “subject” and “patient” are used interchangeably. A subject can be a mammal such as a non-primate (e.g., cows, pigs, horses, cats, dogs, goats, rabbits, rats, mice, etc.) or a primate (e.g., monkey and human), for example a human. In certain embodiments, the subject is a mammal, e.g., a human, diagnosed with a disease or disorder provided herein.

In another embodiment, the subject is a mammal, e.g., a human, at risk of developing a disease or disorder provided herein. In a specific embodiment, the subject is human.

The terms “therapies” and “therapy” are used in their broadest sense understood in the clinical arts.

The term “pharmaceutically acceptable” indicates that the material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables.

The term “carrier” refers to a glidant, diluent, adjuvant, excipient, or vehicle etc. with which the compound is administered, without limitation. Examples of carriers are described herein and also in Remington: The Science and Practice of Pharmacy (Remington: The Science and Practice of Pharmacy, 23rd Edition, ISBN-13: 978-0128200070).

The term “diluent” refers to chemical compounds that are used to dilute the compound of interest prior to delivery. Diluents can also serve to stabilize compounds. Non-limiting examples of diluents include starch, saccharides, disaccharides, sucrose, lactose, polysaccharides, cellulose, cellulose ethers, hydroxypropyl cellulose, sugar alcohols, xylitol, sorbitol, maltitol, microcrystalline cellulose, calcium or sodium carbonate, lactose, lactose monohydrate, dicalcium phosphate, cellulose, compressible sugars, dibasic calcium phosphate dehydrate, mannitol, and tribasic calcium phosphate.

The term “binder” when used herein relates to any pharmaceutically acceptable film which can be used to bind together the active and inert components of the carrier together to maintain cohesive and discrete portions. Non-limiting examples of binders include hydroxypropylcellulose, hydroxypropylmethylcellulose, povidone, copovidone, and ethyl cellulose.

The term “disintegrant” refers to a substance which, upon addition to a solid preparation, facilitates its break-up or disintegration after administration and permits the release of an active ingredient as efficiently as possible to allow for its rapid dissolution.

Non-limiting examples of disintegrants include maize starch, sodium starch glycolate, croscarmellose sodium, modified corn starch, sodium carboxymethyl starch, crospovidone, pregelatinized starch, and alginic acid.

The term “lubricant” refers to an excipient which is added to a powder blend to prevent the compacted powder mass from sticking to the equipment during the tableting or encapsulation process. It aids the ejection of the tablet form the dies, and can improve powder flow. Non-limiting examples of lubricants include magnesium stearate, stearic acid, silica, fats, calcium stearate, polyethylene glycol, sodium stearyl fumarate, or talc; and solubilizers such as fatty acids including lauric acid, oleic acid, and C8/C10 fatty acid.

The term “film coating” refers to a thin, uniform, film on the surface of a substrate (e.g., tablet). Film coatings are particularly useful for protecting the active ingredient from photolytic degradation. Non-limiting examples of film coatings include polyvinylalcohol based, hydroxyethylcellulose, hydroxypropylmethylcellulose, sodium carboxymethylcellulose, polyethylene glycol 4000 and cellulose acetate phthalate film coatings.

The term “glidant” as used herein is intended to mean agents used in tablet and capsule formulations to improve flow-properties during tablet compression and to produce an anti-caking effect. Non-limiting examples of glidants include colloidal silicon dioxide, talc, fumed silica, starch, starch derivatives, and bentonite.

The term “effective amount” or “therapeutically effective amount” refers to an amount that is sufficient to effect treatment, as defined herein, when administered to a mammal in need of such treatment. The therapeutically effective amount will vary depending upon the patient being treated, the weight and age of the patient, the severity of the disease condition, the manner of administration and the like, which can readily be determined by one of ordinary skill in the art.

The term “unit dosage forms” or “pharmaceutical dosage forms” refers to physically discrete units suitable as unitary dosages for human patients and other mammals, each unit containing a predetermined quantity of active material calculated to produce the desired therapeutic effect, in association with a suitable pharmaceutical excipient (e.g., a tablet).

The term “treatment” or “treating,” to the extent it relates to a disease or condition includes preventing the disease or condition from occurring, inhibiting the disease or condition, eliminating the disease or condition, and/or relieving one or more symptoms of the disease or condition.

The term “% w/w” as used herein refers to the weight of a component based on the total weight of a composition comprising the component. For example, if component A is present in an amount of 50% w/w in a 100 mg composition, component A is present in an amount of 50 mg.

Unless specifically stated otherwise, where a compound may assume alternative tautomeric, regioisomeric and/or stereoisomeric forms, all alternative isomers, are intended to be encompassed within the scope of the claimed subject matter. For example, when a compound is described as a particular optical isomer D- or L-, it is intended that both optical isomers be encompassed herein. For example, where a compound is described as having one of two tautomeric forms, it is intended that both tautomers be encompassed herein. Thus, the compounds provided herein may be enantiomerically pure, or be stereoisomeric or diastereomeric mixtures. The compounds provided herein may contain chiral centers. Such chiral centers may be of either the (R) or (S) configurations, or may be a mixture thereof. The chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

The present disclosure also encompasses all suitable isotopic variants of the compounds according to the present disclosure, whether radioactive or not. An isotopic variant of a compound according to the present disclosure is understood to mean a compound in which at least one atom within the compound according to the present disclosure has been exchanged for another atom of the same atomic number, but with a different atomic mass than the atomic mass which usually or predominantly occurs in nature. Examples of isotopes which can be incorporated into a compound according to the present disclosure are those of hydrogen, carbon, nitrogen, oxygen, fluorine, chlorine, bromine and iodine, such as ²H (deuterium), ³H (tritium), ¹³C, ¹⁴C, ¹⁵N, ¹⁷O, ¹⁸O, ¹⁸F, ³⁶Cl, ⁸²Br, ¹²³I, ¹²⁴I, ¹²⁵I, ¹²⁹I and ¹³¹I. Particular isotopic variants of a compound according to the present disclosure, especially those in which one or more radioactive isotopes have been incorporated, may be beneficial, for example, for the examination of the mechanism of action or of the active compound distribution in the body. Compounds labelled with ³H, ¹⁴C and/or ¹⁸F isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required. In some embodiments, hydrogen atoms of the compounds described herein may be replaced with deuterium atoms. In certain embodiments, “deuterated” as applied to a chemical group and unless otherwise indicated, refers to a chemical group that is isotopically enriched with deuterium in an amount substantially greater than its natural abundance. Isotopic variants of the compounds according to the present disclosure can be prepared by various, including, for example, the methods described below and in the working examples, by using corresponding isotopic modifications of the particular reagents and/or starting compounds therein.

Thus, any of the embodiments described herein are meant to include a salt, a single stereoisomer, a mixture of stereoisomers and/or an isotopic form of the compounds.

Unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 10%, 9%, 8%, 7%, 6%, 5%, 4%, 3%, 2%, 1%, 0.5%, 0.4%, 0.3%, 0.25%, 0.2%, 0.1% or 0.05% of a given value or range. Unless otherwise specified, the term “about” means within plus or minus 10% of a the explicitly recited value, rounded either up or down to the nearest integer.

### 5. ADDITIONAL EMBODIMENTS

The disclosure is further described by the following non-limiting clauses:

Clause 1. A pharmaceutical composition comprising:
40-60% w/w ebselen;
20-40% w/w filler;
2-9% disintegrant;
0.1-1% lubricant; and
0.1-1% glidant,
wherein the composition is formulated for oral administration.

Clause 2. The pharmaceutical composition of clause 1, comprising 45-60% w/w ebselen.

Clause 3. The pharmaceutical composition of clause 2, comprising 50-60% w/w ebselen.

Clause 4. The pharmaceutical composition of any one of clauses 1 to 3, wherein the filler is present in an amount of 25-40% w/w.

Clause 5. The pharmaceutical composition of clause 4, wherein the filler is present in an amount of 34-40%.

Clause 6. The pharmaceutical composition of clause 4 or 5, wherein the filler is microcrystalline cellulose.

Clause 7. The pharmaceutical composition of any one of clauses 1 to 6, wherein the disintegrant is present in an amount of 4-8% w/w.

Clause 8. The pharmaceutical composition of clause 7, where in the disintegrant is present in an amount of about 4% w/w.

Clause 9. The pharmaceutical composition of clause 7 or 8, wherein the disintegrant is croscarmellose sodium.

Clause 10. The pharmaceutical composition of any one of clauses 1 to 9, wherein the lubricant is present in an amount of 0.25-0.5% w/w.

Clause 11. The pharmaceutical composition of clause 10, wherein the lubricant is magnesium stearate.

Clause 12. The pharmaceutical composition of any one of clauses 1 to 11, wherein the glidant is present in an amount of 0.3 to 0.5% w/w.

Clause 13. The pharmaceutical composition of clause 12, wherein the glidant is colloidal silicon dioxide.

Clause 14. A pharmaceutical dosage form comprising the pharmaceutical composition of any one of clauses 1 to 13.

Clause 15. The pharmaceutical dosage form of clause 14, wherein the ebselen is present in an amount of about 200 mg.

Clause 16. The pharmaceutical dosage form of clause 14, wherein the ebselen is present in an amount of about 400 mg.

Clause 17. The pharmaceutical dosage form of any one of clauses 14 to 16, wherein the dosage form is a solid dosage form.

Clause 18. The pharmaceutical dosage form of clause 17, wherein the solid dosage form is a capsule.

Clause 19. The pharmaceutical dosage form of clause 18, wherein the capsule is an opaque hard gelatin capsule.

Clause 20. The pharmaceutical dosage form of clause 17, wherein the solid dosage form is a tablet.

Clause 21. The pharmaceutical dosage form of clause 20, wherein the tablet further comprises a coating.

Clause 22. The pharmaceutical dosage form of clause 20, wherein the tablet is an uncoated pressed tablet.

Clause 23. The pharmaceutical dosage form of any one of clauses 20 to 22, comprising:
(i) an intra-granular phase comprising:
ebselen,
filler,
disintegrant, and
lubricant; and
(ii) an extra-granular phase comprising:
an additional lubricant, and
glidant.

Clause 24. The pharmaceutical dosage form of any one of clauses 20 to 23, wherein the tablet comprises:
400 mg ebselen,
265 mg microcrystalline cellulose,
28 mg croscarmellose sodium,
3.5 mg magnesium stearate, and
3.5 mg colloidal silicon dioxide.

Clause 25. The pharmaceutical dosage form of clause 24, wherein:
(i) the intra-granular phase comprises 400 mg ebselen, 265 mg microcrystalline cellulose, 28 mg croscarmellose sodium, and 1.75 mg magnesium stearate; and/or
(ii) the extra-granular phase comprises 1.75 mg magnesium stearate and 3.5 mg colloidal silicon dioxide.

Clause 26. The pharmaceutical dosage form of any one of clauses 20 to 25, wherein the tablet is formed by dry granulation.

Clause 27. The pharmaceutical dosage form of any one of clauses 14 to 26, wherein the dosage form has a disintegration time of two hours or less and a dissolution rate of 70% or more at 30 minutes.

Clause 28. The pharmaceutical dosage form of clause 27, wherein the dosage form has a disintegration time of one hour or less and a dissolution rate of 80% or more at 30 minutes.

Clause 29. The pharmaceutical dosage form of any one of clauses 14 to 26,
wherein oral administration of said pharmaceutical dosage form to a selected human subject group produces in said selected human subject group:
a maximum blood plasma concentration (average Cₘₐₓ) that is greater than that achieved with a control ebselen formulation;
a maximum blood plasma concentration (average Cₘₐₓ) that is achieved within 4 hours or less of administration.

Clause 30. A method of delivering a therapeutically effective amount of ebselen to a subject in need thereof, comprising orally administering to a subject in need thereof a pharmaceutical dosage form according to any one of clauses 14 to 28, to achieve:
an enhanced maximum blood plasma concentration (Cₘₐₓ) for ebselen that is greater than that achieved with a control ebselen formulation; and/or
a maximum blood plasma concentration (Cₘₐₓ) for ebselen that is achieved within 4 hours or less of administration.

Clause 31. The method of clause 30, wherein the pharmaceutical dosage form is administered twice daily.

Clause 32. The method of clause 30 or 31, wherein the pharmaceutical dosage form comprises 400 mg ebselen, and oral administration is performed twice daily.

Clause 33. The method of clause 30 or 31, wherein the pharmaceutical dosage form comprises 800 mg ebselen, and oral administration is performed twice daily.

Clause 34. The method of any one of clauses 30 to 33, wherein the maximum blood plasma concentration (Cₘₐₓ) achieved after one (1) day is at least about 1.2-fold greater than the Cₘₐₓ achieved with a control ebselen formulation.

Clause 35. The method of clause 34, wherein the maximum blood plasma concentration (Cₘₐₓ) achieved after one (1) day is at least about 1.5-fold greater than the Cₘₐₓ achieved with a control ebselen formulation.

Clause 36. The method of any one of clauses 30 to 33, wherein the dosage form is orally administered as a single tablet of 400 mg ebselen and exhibits greater mean peak concentration (Cₘₐₓ) as compared to a dosage form administered as two capsules each comprising 200 mg ebselen, 120 mg microcrystalline cellulose, 28 mg croscarmellose sodium and 1.8 mg magnesium stearate.

Clause 37. An uncoated pressed tablet comprising:
400 mg ebselen,
265 mg microcrystalline cellulose,
28 mg croscarmellose sodium,
3.5 mg magnesium stearate, and
3.5 mg colloidal silicon dioxide.

Clause 38. The uncoated pressed tablet of clause 37, wherein the tablet comprises:
(i) an intra-granular phase comprising 400 mg ebselen, 265 mg microcrystalline cellulose, 28 mg croscarmellose sodium, and 1.75 mg magnesium stearate; and
(ii) an extra-granular phase comprises 1.75 mg magnesium stearate and 3.5 mg colloidal silicon dioxide.

### 6. EXAMPLES

The Examples in this section are offered by way of illustration, and not by way of limitation. The examples can represent only some embodiments, and it should be understood that the following examples are illustrative and not limiting. All substituents, unless otherwise specified, are as previously defined. The reagents and starting materials are readily available to one of ordinary skill in the art. The specific synthetic steps for each of the routes described may be combined in different ways, or in conjunction with steps from different schemes, to prepare the compounds described herein.

### 6.1. Example 1: Preparation of Control Ebselen Dosage Form (200 mg Capsule) (“Formulation 1”)

The following control ebselen dosage is a gelatin capsule containing 200 mg of ebselen and 150 mg of excipients microcrystalline cellulose, sodium croscarmellose and magnesium stearate, and has been previously described (see for example, U.S. patent application Ser. No. 16/300,935 (published as US20200261417)).

The quantitative composition of control ebselen dosage from, 200 mg capsules, is described in Table 1.

TABLE 1
Quantitative composition of control
ebselen dosage form (Capsule, 200 mg)

| Material | Function | Capsules (mg) | % w/w |
|---|---|---|---|
| Ebselen | Drug | 200.0 | 47.3 |
| | Substance | | |
| Microcrystalline cellulose | Filler | 120.2 | 28.3 |
| Croscarmellose Sodium | Disintegrant | 28.0 | 6.6 |
| Magnesium Stearate | Lubricant | 1.8 | 0.4 |
| White Opaque Hard Gelatin | Capsule Shell | 73.7 | 17.4 |
| Capsule, Size #1 | | | |
| Target Capsule Weight | | 423.7 | 100.0 |

Components except magnesium stearate are individually weighed, manually screened, and then blended. Magnesium stearate is screened, added to the blender and mixed with the previously blended components. The resulting blend is filled into the capsule shells to the appropriate fill weight.

Blend was discharged into low density polyethylene bags labeled and transferred to the encapsulator for encapsulation in gelatin capsules.

The control ebselen dosage form was obtained as capsules containing 200 mg of ebselen.

### 6.2. Example 2: Preparation of Ebselen Tablet (400 mg) (“Formulation 2”)

To investigate reducing the number of dosage units of ebselen, a dosage form with an increased unit dose concentration of ebselen was formulated. As such a solid oral formulation in the form of capsule or tablet, each containing ebselen, 400 mg was investigated. Initial feasibility batches were evaluated for both capsule and tablet dosage forms.

Feasibility development was initiated with a dry granulation procedure with projected fill weight of 700 mg/capsule for encapsulation of ebselen 400 mg capsules. Size “00” capsules were evaluated for encapsulation of ebselen 400 mg capsules. The same blend was utilized for the tableting evaluation of ebselen 400 mg tablets.

Initial feasibility batches of ebselen 400 mg tablets and capsules were executed with the same percentage unit composition of the earlier control ebselen dosage form (200 mg capsules, see e.g., Example 1 above).

During early stages of development, the tablet seemed feasible, with a smaller compressed dimensional size compared to the capsule form (size 00). As such, development work was mainly focused on developing a stable tablet formulation for ebselen with 400 mg as an active strength. The dry granulation procedure was selected as a process of choice for manufacturing the 400 mg tablets, to ensure satisfactory tableting parameters and drug release profile.

Croscarmellose sodium (8% w/w) was utilized as disintegrant in the initial feasibility batches. Drug release profile of the initial feasibility batch with 8% w/w croscarmellose sodium was found to be faster than the dissolution profile of the control ebselen dosage form (Formulation 1, Example 1). Hence, a subsequent feasibility batch was examined with 2% w/w croscarmellose sodium. The resulting drug release profile for the batch with 2% w/w croscarmellose sodium was also found to be faster than the dissolution profile of the control ebselen dosage form (Formulation 1, Example 1).

Prototype stability batches were manufactured using lab model roll compactor and the samples comprising 8% w/w croscarmellose sodium and 2% w/w croscarmellose sodium were stored at 40° C./75% RH (accelerated storage) for 1 month and tested for dissolution, assay, water content and impurity profiles. Stability levels were found to be satisfactory for both samples comprising different amounts of disintegrant (i.e., 8% w/w croscarmellose sodium and 2% w/w croscarmellose sodium).

Surprisingly, all of the tablets with 8% croscarmellose as disintegrant showed cracks on the surface of the tablet when open exposed at accelerated condition (40° C./75% relative humidity (RH)) within 2 weeks. By contrast, with respect to tablets with 2% croscarmellose as disintegrant only 1 tablet out of 10 showed cracks on the surface of the tablet when open exposed at accelerated condition (40° C./75% relative humidity (RH)) within 4 weeks.

Next, a tablet was evaluated comprising 400 mg of ebselen and 4% w/w croscarmellose sodium as disintegrant, manufactured by a dry granulation procedure using the same equipment design proposed for clinical batch manufacturing. A non-GMP scale-up batch was executed with 4% w/w croscarmellose sodium as disintegrant by dry granulation procedure to assess the impact of scale-up on processability with higher batch size. Quantitative composition of uncoated pressed ebselen tablets is described in Table 2.

TABLE 2
Composition of Formulation 2: Ebselen Tablets, 400 mg

| | Product Name | |
|---|---|---|
| | Ebselen Tablet 400 mg | |
| Material Name | mg/tablet | % w/w |
| Intra-granular | | |
| Ebselen | 400.00 | 57.14 |
| Microcrystalline Cellulose PH-102 | 265.00 | 37.86 |
| Croscarmellose Sodium SD-711 | 28.00 | 4.00 |
| Magnesium Stearate LIGAMED MF-2-V | 1.75 | 0.25 |
| Extra-granular | | |
| Magnesium Stearate LIGAMED MF-2-V | 1.75 | 0.25 |
| Colloidal silicon dioxide | 3.50 | 0.50 |
| Aerosil 200 Pharma | | |
| Total | 700.00 | 100.00 |

Manufacturing processability, tableting properties and drug release profile were found satisfactory for Formulation 2 (ebselen uncoated pressed tablet, 400 mg). Non-GMP scale-up batch manufacturing was examined with the same equipment design and manufacturing process as proposed for clinical batch manufacturing. Samples from this scale-up batch were packaged in high density polyethylene (HDPE) bottles (30 tablets/bottle) and induction sealed bottles were subsequently stored at 25° C./60% RH (long term storage), 30° C./65% RH (intermediate storage) and 40° C./75% RH (accelerated storage).

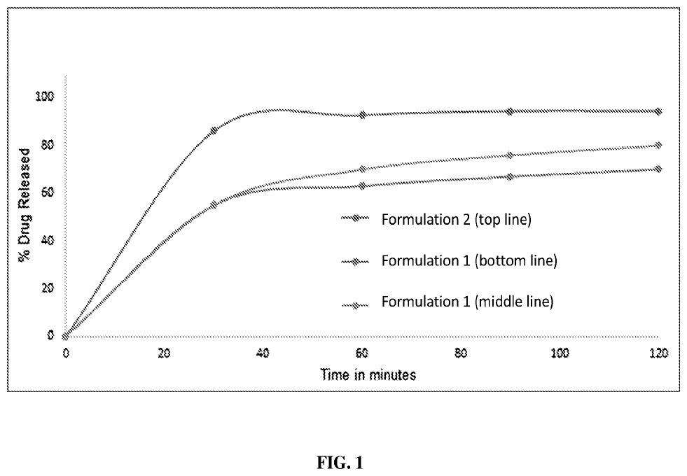

In summary, appearance, assay, related substances, dissolution profile and water content of Formulation 2 were monitored as per stability protocol and found to be stable and suitable for further development. As shown in FIG. 1, Formulation 2 (ebselen tablet 400 mg, top line) has a faster dissolution profile/drug release rate that Formulation 1 (a control ebselen dosage from, capsule 200 mg, bottom two lines).

### 6.3. Example 3: Cross-Over Plasma Pharmacokinetic Study of Ebselen Following Oral Administration of 1×400 mg Tablet BID Versus 2× Control Ebselen 200 mg Capsules BID to Naïve Bama Minipigs

Method: Three male and three female naïve Bama mini-pigs were used in this study. The study was conducted in three “phases”. In phase 1, mini-pigs were dosed with control ebselen capsules (one animal, MP1502, vomited the capsules). In phase 2, mini-pigs were dosed with the subject tablets (e.g., 400 mg ebselen tablets). In phase 3, oral dosing with capsules was repeated for animal MP1502, replaced by animal MP1503 (“phase 3”). Animals in Phases 1 and 3 were orally administered control ebselen capsules, (2×200 mg each) (“Formulation 1”) twice daily (BID, 400 mg/animal/dose, 2×200 mg/capsule). Animals in Phase 2 were orally administered the subject ebselen tablet, 400 mg (“Formulation 2”) twice daily (BID, 400 mg/animal/dose, 1×400 mg/tablet). There was one week wash out period between last dosing day of Phase 1 and first dosing day of Phase 2. Blood samples were collected at 0.5, 1, 2, 3, 4, 6, 8 and 12 hours post the first and fifth dosing. Concentrations of ebselen and ebselen glucuronide in plasma samples were determined by a liquid chromatography tandem mass spectrometry (LC-MS/MS) method.

Results: Surprisingly, ebselen levels for formulation 2 exhibited greater Cₘₐₓ compared to formulation 1 (Table 3).

TABLE 3
Ebselen levels for Formulation 2 exhibited greater Cₘₐₓ
compared to Formulation 1
Ebselen levels in male and female Bama Minipigs following oral
administration of Formulation1 and Formulation2, BID Day 1,
BID Day 2 and QD Day 3

| | | | Cₘₐₓ Ratio |
|---|---|---|---|
| | | Cₘₐₓ (Formulation2 | (Formulation2/ |
| Day | Gender | and Formulation1) | Formulation1) |
| 1.00 | Male | 1248/713 | 1.75 |
| | Female | 637/564 | 1.13 |
| | Mean Male and Female | 943/639 | 1.48 |
| 3.00 | Male | 2350/2213 | 1.06 |
| | Female | 941/588 | 1.60 |
| | Mean Male and Female | 1646/1401 | 1.17 |

In summary, ebselen levels for Formulation 2 exhibited greater Cₘₐₓ compared to Formulation 1.

### 7. EQUIVALENTS AND INCORPORATION BY REFERENCE

While the disclosure has been particularly shown and described with reference to a preferred embodiment and various alternate embodiments, it will be understood by persons skilled in the relevant art that various changes in form and details can be made therein without departing from the spirit and scope of the disclosure.

All publications, patents, patent applications, and other documents cited in this application, including U.S. Provisional Appl. No. 63/349,470, are hereby incorporated by reference in their entireties for all purposes to the same extent as if each individual publication, patent, patent application, or other document were individually indicated to be incorporated by reference for all purposes.

What is claimed is:

1. A pharmaceutical composition comprising:
40-60% w/w ebselen;
20-40% w/w filler;
2-9% disintegrant;
0.1-1% lubricant; and
0.1-1% glidant,
wherein the composition is formulated for oral administration.

2. The pharmaceutical composition of claim 1, comprising 45-60% w/w ebselen.

3. The pharmaceutical composition of claim 2, comprising 50-60% w/w ebselen.

4. The pharmaceutical composition of any one of claims 1 to 3, wherein the filler is present in an amount of 25-40% w/w.

5. The pharmaceutical composition of claim 4, wherein the filler is present in an amount of 34-40%.

6. The pharmaceutical composition of claim 4 or 5, wherein the filler is microcrystalline cellulose.

7. The pharmaceutical composition of any one of claims 1 to 6, wherein the disintegrant is present in an amount of 4-8% w/w.

8. The pharmaceutical composition of claim 7, where in the disintegrant is present in an amount of about 4% w/w.

9. The pharmaceutical composition of claim 7 or 8, wherein the disintegrant is croscarmellose sodium.

10. The pharmaceutical composition of any one of claims 1 to 9, wherein the lubricant is present in an amount of 0.25-0.5% w/w.

11. The pharmaceutical composition of claim 10, wherein the lubricant is magnesium stearate.

12. The pharmaceutical composition of any one of claims 1 to 11, wherein the glidant is present in an amount of 0.3 to 0.5% w/w.

13. The pharmaceutical composition of claim 12, wherein the glidant is colloidal silicon dioxide.

14. A pharmaceutical dosage form comprising the pharmaceutical composition of any one of claims 1 to 13.

15. The pharmaceutical dosage form of claim 14, wherein the ebselen is present in an amount of about 200 mg.

16. The pharmaceutical dosage form of claim 14, wherein the ebselen is present in an amount of about 400 mg.

17. The pharmaceutical dosage form of any one of claims 14 to 16, wherein the dosage form is a solid dosage form.

18. The pharmaceutical dosage form of claim 17, wherein the solid dosage form is a capsule.

19. The pharmaceutical dosage form of claim 18, wherein the capsule is an opaque hard gelatin capsule.

20. The pharmaceutical dosage form of claim 17, wherein the solid dosage form is a tablet.

21. The pharmaceutical dosage form of claim 20, wherein the tablet further comprises a coating.

22. The pharmaceutical dosage form of claim 20, wherein the tablet is an uncoated pressed tablet.

23. The pharmaceutical dosage form of any one of claims 20 to 22, comprising:
(i) an intra-granular phase comprising:
ebselen,
filler,
disintegrant, and
lubricant; and
(ii) an extra-granular phase comprising:
an additional lubricant, and
glidant.

24. The pharmaceutical dosage form of any one of claims 20 to 23, wherein the tablet comprises:
400 mg ebselen,
265 mg microcrystalline cellulose,
28 mg croscarmellose sodium,
3.5 mg magnesium stearate, and
3.5 mg colloidal silicon dioxide.

25. The pharmaceutical dosage form of claim 24, wherein:
(i) the intra-granular phase comprises 400 mg ebselen, 265 mg microcrystalline cellulose, 28 mg croscarmellose sodium, and 1.75 mg magnesium stearate; and
(ii) the extra-granular phase comprises 1.75 mg magnesium stearate and 3.5 mg colloidal silicon dioxide.

26. The pharmaceutical dosage form of any one of claims 20 to 25, wherein the tablet is formed by dry granulation.

27. The pharmaceutical dosage form of any one of claims 14 to 26, wherein the dosage form has a disintegration time of two hours or less and a dissolution rate of 70% or more at 30 minutes.

28. The pharmaceutical dosage form of claim 27, wherein the dosage form has a disintegration time of one hour or less and a dissolution rate of 80% or more at 30 minutes.

29. The pharmaceutical dosage form of any one of claims 14 to 26, wherein oral administration of said pharmaceutical dosage form to a selected human subject group produces in said selected human subject group:
a maximum blood plasma concentration (average Cₘₐₓ) that is greater than that achieved with a control ebselen formulation; and
a maximum blood plasma concentration (average Cₘₐₓ) that is achieved within 4 hours or less of administration.

30. A method of delivering a therapeutically effective amount of ebselen to a subject in need thereof, comprising orally administering to a subject in need thereof a pharmaceutical dosage form according to any one of claims 14 to 28, to achieve:
an enhanced maximum blood plasma concentration (Cₘₐₓ) for ebselen that is greater than that achieved with a control ebselen formulation; and
a maximum blood plasma concentration (Cₘₐₓ) for ebselen that is achieved within 4 hours or less of administration.

31. The method of claim 30, wherein the pharmaceutical dosage form is administered twice daily.

32. The method of claim 30 or 31, wherein the pharmaceutical dosage form comprises 400 mg ebselen, and oral administration is performed twice daily.

33. The method of claim 30 or 31, wherein the pharmaceutical dosage form comprises 800 mg ebselen, and oral administration is performed twice daily.

34. The method of any one of claims 30 to 33, wherein the maximum blood plasma concentration (Cₘₐₓ) achieved after one (1) day is at least about 1.2-fold greater than the Cₘₐₓ achieved with a control ebselen formulation.

35. The method of claim 34, wherein the maximum blood plasma concentration (Cₘₐₓ) achieved after one (1) day is at least about 1.5-fold greater than the Cₘₐₓ achieved with a control ebselen formulation.

36. The method of any one of claims 30 to 33, wherein the dosage form is orally administered as a single tablet of 400 mg ebselen and exhibits greater mean peak concentration (Cₘₐₓ) as compared to a dosage form administered as two capsules each comprising 200 mg ebselen, 120 mg microcrystalline cellulose, 28 mg croscarmellose sodium and 1.8 mg magnesium stearate.

37. An uncoated pressed tablet comprising:
400 mg ebselen,
265 mg microcrystalline cellulose,
28 mg croscarmellose sodium,
3.5 mg magnesium stearate, and
3.5 mg colloidal silicon dioxide.

38. The uncoated pressed tablet of claim 37, wherein the tablet comprises:
(i) an intra-granular phase comprising 400 mg ebselen, 265 mg microcrystalline cellulose, 28 mg croscarmellose sodium, and 1.75 mg magnesium stearate; and
(ii) an extra-granular phase comprises 1.75 mg magnesium stearate and 3.5 mg colloidal silicon dioxide.
